(19) United States
(12) Patent Application Publication
(10) Pub. No.: US 2024/0207433 A1
(43) Pub. Date: Jun. 27, 2024

(54) DRUG CONJUGATES CONTAINING ALPHA-ENOLASE ANTIBODIES AND USES THEREOF

(71) Applicant: HUNILIFE BIOTECHNOLOGY, INC., Taipei (TW)

(72) Inventors: Ta-Tung Yuan, Taipei (TW); Chi-Kuan Chen, Taipei (TW); Mao-Lin Chen, Taipei (TW)

(73) Assignee: HUNILIFE BIOTECHNOLOGY, INC., Taipei (TW)

(21) Appl. No.: 17/998,559

(22) PCT Filed: May 10, 2021
(86) PCT No.: PCT/CN2021/092811

§ 371 Date: Nov. 11, 2022

Related U.S. Application Data
(60) Provisional application No. 63/022,702, filed on May 11, 2020.

(51) Int. Cl. A61K 47/68; A61K 45/06; A61P 11/00; A61P 35/00; A61P 37/06; C07K 16/40

(52) U.S. Cl. CPC A61K 47/6871; A61K 45/06; A61P 11/00; A61P 35/00; A61P 37/06; C07K 16/40

(57) ABSTRACT

An immunoconjugate includes an anti-ENO-1 antibody, or a binding fragment thereof, and a therapeutic agent or a label, having the formula: Ab-(L-D)ₘ, wherein Ab is the anti-ENO-1 antibody or the binding fragment thereof, L is a linker or a direct bond, D is the therapeutic agent or the label, and m is an integer from 1 to 12. The antibody may be a monoclonal antibody, which may be a humanized antibody or fully human antibody. A method for treating an inflammatory disease, immune disorder, or cancer includes administering to a subject in need of such treatment a pharmaceutically effective amount of an immunoconjugate containing an antibody against ENO-1, or a binding fragment thereof, and a therapeutic agent covalently conjugated with the antibody.

### CROSS-REFERENCE TO RELATED APPLICATION

This application claims the benefit of U.S. Provisional Application No. 63/022,702, filed May 11, 2020, which is hereby incorporated by reference herein for all purposes.

### BACKGROUND OF INVENTION

### Field of the Invention

The present invention relates to antibody-drug conjugates containing anti-human alpha-enolase protein (ENO-1) antibodies and their uses in therapy. The present invention also relates to methods for treating an inflammatory disease or an immune disorder, or suppressing tumor growth and metastasis by administrating an anti-ENO1 ADC to a subject.

### Background Art

Antibody-drug conjugates (ADCs) can provide targeted therapy to treat various diseases or conditions, such as cancer. ADCs are complex molecules comprising antibodies linked to biologically active agents, such as cytotoxic agents or drugs. By combining unique targeting of the antibodies with the therapeutic effects of the drugs, antibody-drug conjugates can distinguish between normal and cancer cells, thereby minimizing the side effects.

ADC typically comprises a cytotoxic drug (e.g. a tubulin inhibitor or a DNA alkylating agent) coupled to an antibody that specifically targets a marker, e.g., a tumor marker. Antibodies track these proteins down in the body and attach themselves to the surface of cancer cells. The binding between the antibody and the target protein (antigen) triggers a signal in the tumor cell, which then internalizes the ADC. After the ADC is internalized, the cytotoxic drug may be released and kills the cancer. Due to the specific targeting, the drug has lower side effects.

Alpha-enolase (enolase-1, ENO-1) is a multiple functional protein, which was first found as a key enzyme of the glycolysis pathways. Under normal conditions. ENO-1 is expressed in the cytosol. However, ENO-1 is also found to express on the cell surfaces of many cancer cells as a plasminogen receptor and on activated hematopoietic cells, such as neutrophils, lymphocytes and monocytes. It is known that the up-regulation of plasminogen receptor proteins can induce a cascade response of the urokinase plasminogen activation system and results in extracellular matrix degradation. As a consequence, it results in increased metastasis of cancer cells and infiltration of immune cells. Inflammatory stimuli, for example LPS, upregulate ENO-1 cell-surface expression on human blood monocytes and U937 monocytic cells by post translational modification and translocation to cell surface.

It is believed the translocation of ENO-1 is regulated by the MAP kinase signal transduction pathway. This implies that increases in the expression of ENO-1 on cell surface may play an important role in the inflammatory diseases. Auto-antibodies against ENO-1 have been found in variable autoimmune and inflammatory diseases, including Lupus erythematosus, systemic sclerosis, Behcet's disease, ulcerative, and Crohn's disease. It has been known that ENO-1, by way of its plasminogen receptor activity, plays a key role in the disease progression of rheumatoid arthritis patients by increasing invasion activities of monocytes and macrophages.

In sum, monocytes with their up-regulated ENO-1 expression on cell surfaces as plasminogen receptors to increase invasion activities are very important for the disease progression of multiple sclerosis, rheumatoid arthritis, and related immune disorders. Therefore, targeting ENO-1 on the cell surface of monocytes has a good potential to treat inflammatory diseases, such as multiple sclerosis, rheumatoid arthritis, Crohn's disease, ulcerative colitis, and systemic Lupus erythematosus, or related immune disorders, such as chronic obstructive pulmonary disease (COPD), asthma, allergy, psoriasis, type 1 diabetes mellitus, atherosclerosis and osteoporosis.

Besides, ENO-1 expression on cancer cell surfaces as a plasminogen receptor can increase invasion activities of the cancer cells. Therefore, ENO-1 is also a potential target for cancer therapy.

Although antibodies against ENO-1 are useful, there remains a need for improved therapeutic agents using anti-ENO-1 ADCs.

### SUMMARY OF INVENTION

The present invention relates to antibody-drug conjugates containing ENO-1 antibodies and their uses in therapy.

One aspect of the invention relates to immunoconjugates. An immunoconjugate in accordance with one embodiment of the invention includes an anti-ENO-1 antibody, or a binding fragment thereof, and a therapeutic agent or a label, having the formula: Ab-(L-D)ₘ, wherein Ab is the anti-ENO-1 antibody or the binding fragment thereof, L is a linker or a direct bond, D is a therapeutic agent or a label, and m is an integer from 1 to 12.

In accordance with any embodiment of the invention, the Ab may comprise a heavy-chain variable domain having three complementary regions including HCDR1 (GYTFTSCVMN; SEQ ID NO: 1), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6).

In accordance with any embodiment of the invention, the Ab may comprise a heavy-chain variable domain having three complementary regions including HCDR1 (GYTFTSXVMN, wherein X is any amino acid but cysteine; SEQ ID NO: 7), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6).

The linker, L, can be a direct bond, in which the payload, D, is directly linked (conjugated) with the antibody or the binding fragment thereof. A linker can be any linker commonly used in protein modification or conjugation, such as a short peptide (e.g., val-cit), a short organic molecule linker (e.g., SMCC, succinimidyl-4 (N-maleimidomethyl) cyclohexane-1-carboxylate), or the like.

The payload, D, can be a therapeutic agent, such as a cytotoxic agent. Examples of cytotoxic agents that may be used with embodiments of the invention may include a maytansinoid (e.g., DM1 or DM4), monomethyl auristatin E (MMAE), monomethyl auristatin F (MMAF), paclitaxel, or the like.

The payload, D, can be a label or agent for diagnosis or imaging. Example of an imaging agent may include DTPA (Diethylenetriaminepentaacetic acid) or DOTA (1,4,7,10-tetraazacyclododecane-1,4,7,10-tetraacetic acid).

In accordance with some embodiments of the invention, the antibody may be a monoclonal antibody, which may be a humanized antibody or a fully human antibody.

One aspect of the invention relates to methods for diagnosing or imaging cells or a tissue expressing ENO-1. A method in accordance with one embodiment of the invention may comprise administering to a subject an immunoconjugate described above.

In accordance with certain embodiments of the invention, the human ENO-1 protein-related disease or disorder may be any condition arising from aberrant activation or expression of human ENO-1 protein. Examples of such diseases include where human ENO-1 protein aberrantly interacts with its ligands, thereby altering cell-adhesion or cell signaling properties. This alteration in cell adhesion or cell signaling properties can result in neoplastic diseases and/or inflammatory or immune diseases.

One aspect of the invention relates to methods for treating an inflammatory disease or an immune disorder, such as multiple sclerosis, rheumatoid arthritis, Crohn's disease, ulcerative colitis, systemic lupus erythematosus, or relative immune disorders, such as chronic obstructive pulmonary disease (COPD), atopic dermatitis, idiopathic pulmonary fibrosis, nonalcoholic steatohepatitis, asthma, allergy, psoriasis, psoriatic arthritis, type 1 diabetes mellitus, atherosclerosis, osteoporosis, systemic sclerosis, virus induced pneumonia, or macrophage activation syndrome.

One aspect of the invention relates to methods for treating cancers. A method in accordance with one embodiment of the invention may comprise administering to a subject in need of cancer treatment a pharmaceutically effective amount of an immunoconjugate described above. The cancer is a highly ENO-1 expression cancer, such as lung, breast, pancreas, liver, colorectal, and prostate cancers.

One skilled in the art would appreciate that a pharmaceutically effective amount depends on many factors, such as patient conditions, age, disease states, routs of administration, etc., and that such effective amount may be determined based on these factors in routine practice without undue experimentation.

Other aspect of the invention will become apparent with the following description.

### BRIEF DESCRIPTION OF THE DRAWINGS

FIG. 1 shows PLRP-HPLC results of HuL001-SMCC-DM1. Example 7 describes that the conjugation reaction went substantially complete and only residual amounts of anti-ENO-1 antibody and ADCs remained.

FIG. 2 shows PLRP-HPLC results of HuL001-SPP-DM4. Example 7 describes that the conjugation reaction went substantially complete and only residual amounts of anti-ENO-1 antibody and ADCs remained.

FIG. 3 shows HIC results of HuL001-mal-vc-MMAE. Example 7 describes that the conjugation reaction went substantially complete and only residual amounts of anti-ENO-1 antibody and ADCs remained.

FIG. 4 shows PLRP-HPLC results of HuL001-Ph-MMAF. Example 7 describes that the conjugation reaction went substantially complete and only residual amounts of anti-ENO-1 antibody and ADCs remained.

FIG. 5 shows HIC results of HuL001-mal-vc-steroid. Example 7 describes that the conjugation reaction went substantially complete and only residual amounts of anti-ENO-1 antibody and ADCs remained.

FIG. 6 shows LC/MS results of HuL001-SMCC-DM1. The details are described in Example 8.

FIG. 7 shows LC/MS results of HuL001-SPP-DM4. The details are described in Example 8.

FIG. 8 shows reduced LC/MS results of HuL001-mal-vc-MMAE. The details are described in Example 8.

FIG. 9 shows LC/MS results of HuL001-Ph-MMAF. The details are described in Example 8.

FIG. 10 shows reduced LC/MS results of HuL001-mal-vc-steroid. The details are described in Example 8.

FIG. 11 shows in vitro cytotoxicity results of HuL001-SPP-DM4 in LPS-stimulated B cell cancer cell line DHL-4. The details are described in Example 9.

FIG. 12 shows there is no detectable in vitro cytotoxicity of HuL001-SMCC-DM1 in isolated normal human B cells regardless of LPS stimulation. The details are described in Example 9.

FIG. 13 shows there is no detectable in vitro cytotoxicity of HuL001-mal-vc-MMAE in isolated normal human B cell regardless of LPS stimulation. The details are described in Example 9.

FIG. 14 shows superior anti-inflammatory effect of HuL001-mal-vc-steroid, in comparison to HuL001, on TNF-α and CCL2 secretion in LPS-treated human monocyte cell line THP-1. The details are described in Example 10.

FIG. 15 shows in vivo efficacy of HuL001-SMCC-DM1 in PC-3 xenograft prostate cancer model. Treating with HuL001-SMCC-DM1 was able to inhibit the tumor growth compared with the vehicle control group. The detailed procedures were performed as described in Example 11.

FIG. 16 shows in vivo efficacy of HuL001-SMCC-DM1 in C57BL/6 EAE disease model. Treating with HuL001-SMCC-DM1 was able to slow the progression of disease symptoms of EAE mice compared with the PBS control group even COPAXONE® group. The detailed procedures were performed as described in Example 12.

FIG. 17 shows in vivo efficacy of HuL001-SMCC-DM1 in C57BL/6 bleomycin-induced pulmonary fibrosis disease model. Treating with HuL001-SMCC-DM1 was able to attenuate the body weight loss and lung weight gain of pulmonary fibrosis mice compared with the bleomycin-treated group. The detailed procedures were performed as described in Example 13.

### DETAILED DESCRIPTION

### General Definitions

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

The terms “antibody” and “immunoglobulin” are used interchangeably in the broadest sense and include monoclonal antibodies (e.g., full length or intact monoclonal antibodies), polyclonal antibodies, monovalent, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The antibodies can be full-length or can comprise a fragment (or fragments) of the antibody having an antigen-binding portion, including, but not limited to, Fab, F(ab′)₂, Fab′, F(ab)′, Fv, single chain Fv (scFv), bivalent scFv (bi-scFv), trivalent scFv (tri-scFv), Fd, dAb fragment (e.g., Ward et al, Nature, 341:544-546 (1989)), an CDR, diabodies, triabodies, tetrabodies, linear antibodies, single-chain antibody molecules, and multispecific antibodies formed from antibody fragments. Single chain antibodies produced by joining antibody fragments using recombinant methods, or a synthetic linker, are also encompassed by the present invention. Bird et al. Science, 1988, 242:423-426. Huston et al, Proc. Natl. Acad. Sci. USA, 1988, 85:5879-5883.

Antibodies with a variable heavy chain region and a variable light chain region that are at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or about 100% homologous to the variable heavy chain region and variable light chain region of the antibody produced by the reference antibody, and can also bind to ENO-1. Homology can be present at either the amino acid or nucleotide sequence level.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The terms “cancer” and “cancerous” refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

As used herein, “treatment” refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing or decreasing inflammation and/or tissue/organ damage, decreasing the rate of disease progression, amelioration or palliation of the disease state, and remission or improved prognosis. In some embodiments, antibodies of the invention are used to delay development of a disease or disorder.

An “individual” or a “subject” is a vertebrate. In certain embodiments, the vertebrate is a mammal. Mammals include, but are not limited to, farm animals (such as cows), sport animals, pets (such as cats, dogs, and horses), primates, mice and rats. In certain embodiments, the vertebrate is a human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount is also one in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount would be less than the therapeutically effective amount.

The term “therapeutic agent” as used herein refers to a substance that inhibits or prevents the function of cells and/or causes destruction of cells. The term is intended to include radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁰C, and radioactive isotopes of lutetium-177, strontium-89 and samarium (¹⁵³Sm)), immunomodulator, cytotoxic agent, and toxins such as small molecule toxins or enzymatically active toxins of bacterial, fungal, plant or animal origin, including synthetic analogs and derivatives thereof.

A “cytotoxic agent” is a chemical compound useful in the treatment of cancer. Examples of chemotherapeutic agents include maytansinoid 1 (DM1), maytansinoid 4 (DM4), Monomethyl auristatin E (MMAE), Monomethyl auristatin F (MMAF), anthracycline, pyrrolobenzodiazepine, α-amanitin, tubulysin, benzodiazepine, erlotinib (TARCEVA®, Genentech/OSI Pharm.), bortezomib (VELCADE®, Millenium Pharm.), fulvestrant (FASLODEX®, Astrazeneca), sunitinib (SUTENT®, SUl1248, Pfizer), letrozole (FEMARA®, Novartis), imatinib mesylate (GLEEVEC®, Novartis), PTK787/ZK 222584 (Novartis), oxaliplatin (ELOXATIN®, Sanofi), leucovorin, rapamycin (Sirolimus, RAPAMUNE®, Wyeth), lapatinib (TYKERB®, GSK572016, GlaxoSmithKline), lonafarnib (SARASAR®, SCH 66336), sorafenib (NEXAVAR®, BAY43-9006, Bayer Labs.), and gefitinib (IRESSA®, Astrazeneca), AG1478, AG1571 (SU 5271; Sugen), alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine (XELODA®, Roche); and pharmaceutically acceptable salts, acids or derivatives of any of the above.

### Exemplary ADC Linker

Suitable exemplary linkers for the ADC are described in, for example, U.S. Pat. No. 7,595,292 (WO2005/007197). The entire content directed to linkers is hereby incorporated by reference herein. The linker, L, attaches the antibody to a drug moiety through covalent bond(s), not comprising a disulfide group. The linker is a bifunctional or multifunctional moiety which can be used to link one or more therapeutic agent or label (D) and an antibody unit (Ab) to form antibody-drug conjugates (ADCs) of Formula (I). Antibody-drug conjugates (ADCs) can be conveniently prepared using a linker having reactive functionality for binding to the Drug and to the Antibody. A cysteine thiol, or an amine, e.g. N-terminus or amino acid side chain such as lysine, of the antibody (Ab) can form a bond with a functional group of a linker reagent, drug moiety or drug-linker reagent.

The linkers are preferably stable extracellularly. Before transport or delivery into a cell, the antibody-drug conjugate (ADC) is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the therapeutic agent or label moiety. Stability of the ADC may be measured by standard analytical techniques such as mass spectroscopy, HPLC, and the separation/analysis technique LC/MS.

Covalent attachment of the antibody and the therapeutic agent or label moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p 234-242).

Typically, the antibody-drug conjugate compounds comprise a Linker unit between the therapeutic agent or label unit and the antibody unit. In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation.

In some embodiments, the linker is cleavable by a cleaving agent that is present in the intracellular environment (e.g., within a lysosome or endosome or caveolea). The linker can be, e.g., a peptidyl linker that is cleaved by an intracellular peptidase or protease enzyme, including, but not limited to, a lysosomal or endosomal protease. In some embodiments, the peptidyl linker is at least two amino acids long or at least three amino acids long. Cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in 158PID7-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other examples of such linkers are described, e.g., in U.S. Pat. No. 6,214,345, incorporated herein by reference in its entirety and for all purposes. In a specific embodiment, the peptidyl linker cleavable by an intracellular protease is a Val-Cit linker or a Phe-Lys linker (see, e.g., U.S. Pat. No. 6,214,345, which describes the synthesis of doxorubicin with the val-cit linker). One advantage of using intracellular proteolytic release of the therapeutic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond (see, e.g., U.S. Pat. No. 5,622,929).

In yet other embodiments, the linker is cleavable under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker unit is not cleavable and the drug is released by antibody degradation. (See U.S. Publication No. 2005/0238649 incorporated by reference herein in its entirety and for all purposes).

Typically, the linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of antibody-drug conjugate compound, are cleaved when the antibody-drug conjugate compound presents in an extracellular environment (e.g., in plasma). Whether a linker is not substantially sensitive to the extracellular environment can be determined, for example, by incubating with plasma the antibody-drug conjugate compound for a predetermined time period (e.g., 2, 4, 8, 16, or 24 hours) and then quantitating the amount of free drug present in the plasma.

In other, non-mutually exclusive embodiments, the linker promotes cellular internalization. In certain embodiments, the linker promotes cellular internalization when conjugated to the therapeutic agent (i.e., in the milieu of the linker-therapeutic agent moiety of the antibody-drug conjugate compound as described herein).

A variety of exemplary linkers that can be used with the present compositions and methods are described in WO 2004-010957, U.S. Publication No. 2006/0074008, U.S. Publication No. 20050238649, and U.S. Publication No. 2006/0024317 (each of which is incorporated by reference herein in its entirety and for all purposes).

Embodiments of the invention relate to antibody-drug conjugates containing ENO-1 antibodies and their uses in therapy. ENO-1 is a multiple functional protein, which is found to express on the cell surfaces of many cancer cells as a plasminogen receptor and on activated hematopoietic cells, such as neutrophils, lymphocytes and monocytes. Therefore, ADCs based on antibodies against ENO-1 can be useful diagnostic and/or treatment agents.

However, the fast internalization or lacking ADCC activity of therapeutic antibody might result in antibody ineffective as well as resistance. Therefore, there is a need to enhance the therapeutic efficacy of anti-ENO-1 based therapeutics. One approach is to conjugate a payload with an anti-ENO-1 antibody (i.e., an antibody-drug conjugate). By conjugating anti-ENO-1 antibodies to payloads (i.e., ADCs), embodiments of the invention are more potent than the naked anti-ENO-1 antibodies, thereby enabling one to use less antibodies.

In accordance with embodiments of the invention, anti-ENO-1 antibodies, or a binding fragment thereof, may be coupled to a drug, diagnostic agent, or a therapeutic agent. Thus, the term “antibody-drug conjugate” (ADC) as used herein may refer to an antibody portion (which can be a whole antibody or a binding fragment thereof) coupled to a payload (which can be a drug, a diagnostic agent or a therapeutic agent).

The ADCs of the invention contain payloads designed for the therapeutic or diagnostic uses. These ADCs have better biological activities and would require less amounts to achieve the desired effects, as compare with the naked anti-ENO-1 antibodies.

Embodiments of the invention will be illustrated with the following specific examples. One skilled in the art would appreciate that these examples are for illustration only and that other modifications and variations are possible without departing from the scope of the invention.

### Examples

Unless otherwise indicated, each ¹H NMR data were obtained at 500 MHz. The abbreviations used herein are as follows, unless specified otherwise:

Bu: butyl; Bn: benzyl; BOC: t-butyloxycarbonyl; BOP: benzotriazol-1-yloxy tri/dimethylamino-phosphonium hexafluorophosphate; DCC: dicyclohexylcarbodiimide DMF: N,N-dimethylformamide; DMAP: 4-dimethylaminopyridine; EDC: 1-(3-dimethylaminopropyl) 3-ethylcarbodiimide hydrochloride; EtOAc: ethyl acetate; Eq.: equivalent(s); HOBt: hydroxybenztriazole; LAH: lithium aluminum hydride; MeOH: methanol; MHz: megahertz; MS(ES): mass spectrophotometer-electron spray; NMP: N-methylpyrrolidinone; Ph: phenyl; Pr: propyl; TEA: triethylamine; THF: tetrahdrofuran; TLC thin layer chromatography; Tetrakis tetrakis(triphenylphosphine)palladium.

### Example 1. Preparation of Anti-ENO-1 Antibody

In accordance with embodiments of the invention, a general method for the generation of anti-ENO-1 antibodies include obtaining a hybridoma producing a monoclonal antibody against ENO-1. Methods for the production of monoclonal antibodies are known in the art and will not be elaborated here. Briefly, mice are challenged with antigen (ENO-1) with an appropriate adjuvant. Then, the spleen cells of the immunized mice were harvested and fused with hybridoma. Positive clones may be identified for their abilities to bind ENO-1 antigen, using any known methods, such as ELISA. In an embodiment, the anti-ENO-1 antibody is HuL001. Exemplary antibody HuL001 is as described in US2019/0322762, the contents of which are incorporated by reference in its entirety.

Antibody-drug conjugates (ADCs) of the claimed invention can specifically target ENO-1. These ADCs can use any antibody that binds specifically to ENO-1. For example, ADCs of the claimed invention may use a mouse or humanized anti-ENO-1 antibody, or a scFv or Fab fragment thereof. An exemplary anti-ENO-1 antibody, e.g. HuL001, may comprise a heavy-chain variable domain having three complementary regions including HCDR1 (GYTFTSCVMN; SEQ ID NO: 1), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6). An another exemplary anti-ENO-1 antibody may comprise a heavy-chain variable domain having three complementary regions including HCDR1 (GYTFTSXVMN, wherein X is any amino acid but cysteine; SEQ ID NO: 7), HCDR2 (YINPYNDGTKYNEKFKG; SEQ ID NO: 2) and HCDR3 (EGFYYGNFDN; SEQ ID NO: 3), and a light-chain variable domain having three complementary regions including LCDR1 (RASENIYSYLT; SEQ ID NO: 4), LCDR2 (NAKTLPE; SEQ ID NO: 5) and LCDR3 (QHHYGTPYT; SEQ ID NO: 6).

In accordance with embodiments of the invention, the antibodies may be mouse antibodies. Alternatively, the antibodies may be chimeric antibodies (e.g., human constant regions coupled to the mouse variable regions) or humanized antibodies (e.g., mouse CDRs grafted on the framework regions of human immunoglobulins) or completely human antibodies.

The monoclonal antibody may be humanized by obtaining the CDR sequences from the hybridoma and cloning the CDR sequences into human framework sequences to produce humanized antibodies. Any common methods known in the art for identifying CDR sequences may be used. The CDR regions in this invention are identified with the Kabat number scheme. First, a hybridoma of anti-ENO-1 (e.g., mouse hybridoma) was generated. Such a hybridoma may be generated with standard protocols for the production of monoclonal antibodies. The total RNA of the hybridoma was then isolated, for example using the TRIzol® reagent. Then, cDNA was synthesized from the total RNA, for example using a first strand cDNA synthesis kit (Superscript III) and an oligo(dT₂₀) primer or an Ig-3′ constant region primer.

Heavy and light chain variable regions of the immunoglobulin genes were then cloned from the cDNA. For example, the VH and VL variable regions of the anti-ENO-1 mAb were amplified from mouse hybridoma cDNAs by PCR, using a mouse Ig-5′ primer set (Novagen). The PCR products may be cloned directly into a suitable vector (e.g., a pJET1.2 vector) using CloneJet™ PCR Cloning Kit (Ferments). The pJET1.2 vector contains lethal insertions and will survive the selection conditions only when the desired gene is cloned into this lethal region. This facilitates the selection of recombinant colonies. Finally, the recombinant colonies were screened for the desired clones, the DNAs of those clones were isolated and sequenced. The immunoglobulin (IG) nucleotide sequences may be analyzed at the international ImMunoGeneTics information system (IGMT) website.

### Antibody Expression and Purification

For antibody production, the isolated clones may be expressed in any suitable cells. As an example, F293 cells (Life technologies) were transfected with the anti-ENO-1 mAb expressing plasmid and cultured for 7 days. The anti-ENO-1 antibody was purified from the culture medium using a protein A affinity column (GE). Protein concentrations may be determined with a Bio-Rad protein assay kit and analyzed with 12% SDS-PAGE, using procedures known in the art or according to the manufacturer's instructions.

In accordance with embodiments of the invention, any of these anti-ENO-1 antibodies may be used to prepare antibody-drug conjugates (ADCs), as illustrated in the following examples.

### Example 2. Preparation of HuL001-SMCC-DM1 Conjugates

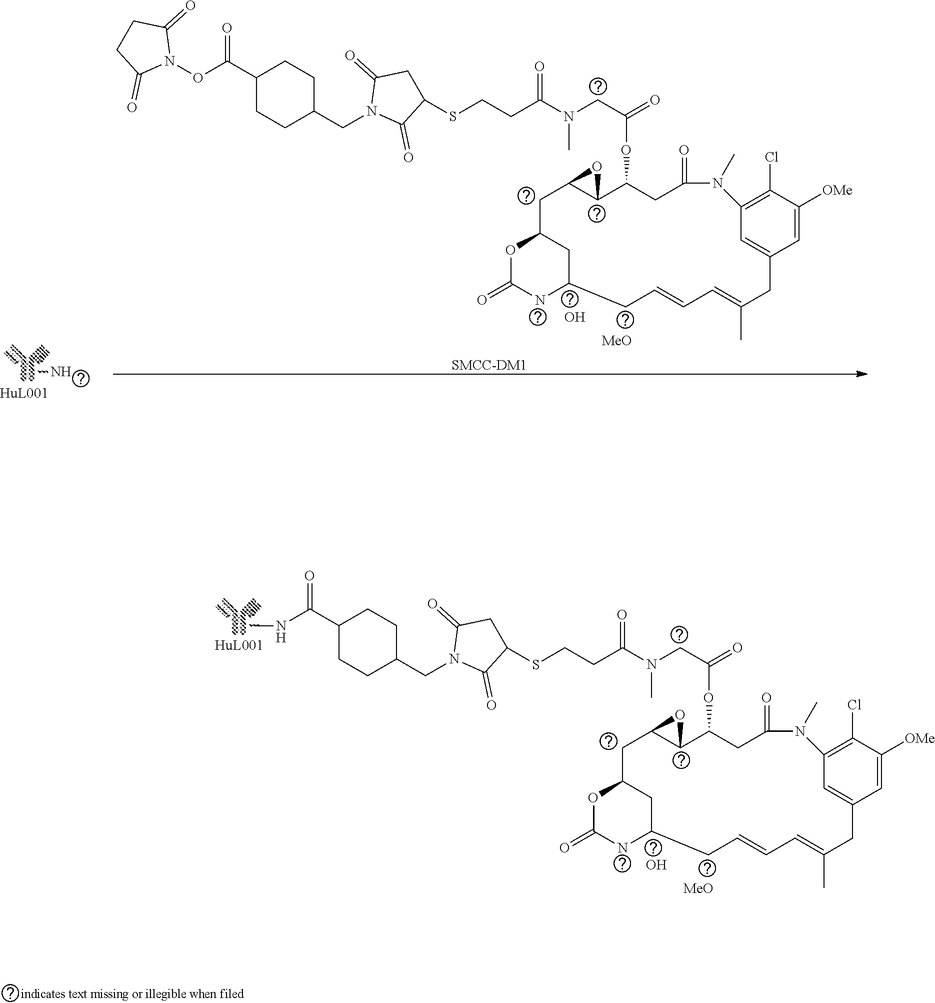

In this example, ADC contains DM1, which is a maytansinoid that was developed for cancer therapy. Maytansine, a benzoansamacrolide, is a highly potent microtubule-targeted compound that induces mitotic arrest and kills tumor cells at subnanomolar concentrations. DM1 binds at the tips of microtubules to suppress the dynamicity of microtubules, i.e., inhibiting the assembly of microtubules. DM1 is a maytansinoid with less systemic toxicity than maytansine. In this example, SMCC-DM1, which is DM1 with a reactive linker SMCC, is used to react with antibody to make antibody drug conjugates. SMCC-DM1 is available from commercial sources, such as MedKoo Biosciences, Inc. or ALB Technology.

For example, the buffer of HuL001 (70 mg) was exchanged to pH 6.5 Na-citrate buffer and adjust to 5 mg/mL. The solution of SMCC-DM1 (5 mM in DMA, 16 eq) was slowly added to the HuL001 solution. The reaction mixture was incubated in shaking incubator (150 rpm) at 37° C. for 18 hours. The Amicon Ultra-15 centrifugal filter with 30 kDa NMWL and 25 mM Na-citrate pH 6.5 was used for the concentration of HuL001-SMCC-DM1 and the removal of SMCC-DM1. ADC concentration: 5.478 mg/mL, ADC yield: 16.5 mg (23.5%), and average DAR: 3.87.

### Example 3. Preparation of HuL001-SPP-DM4 Conjugates

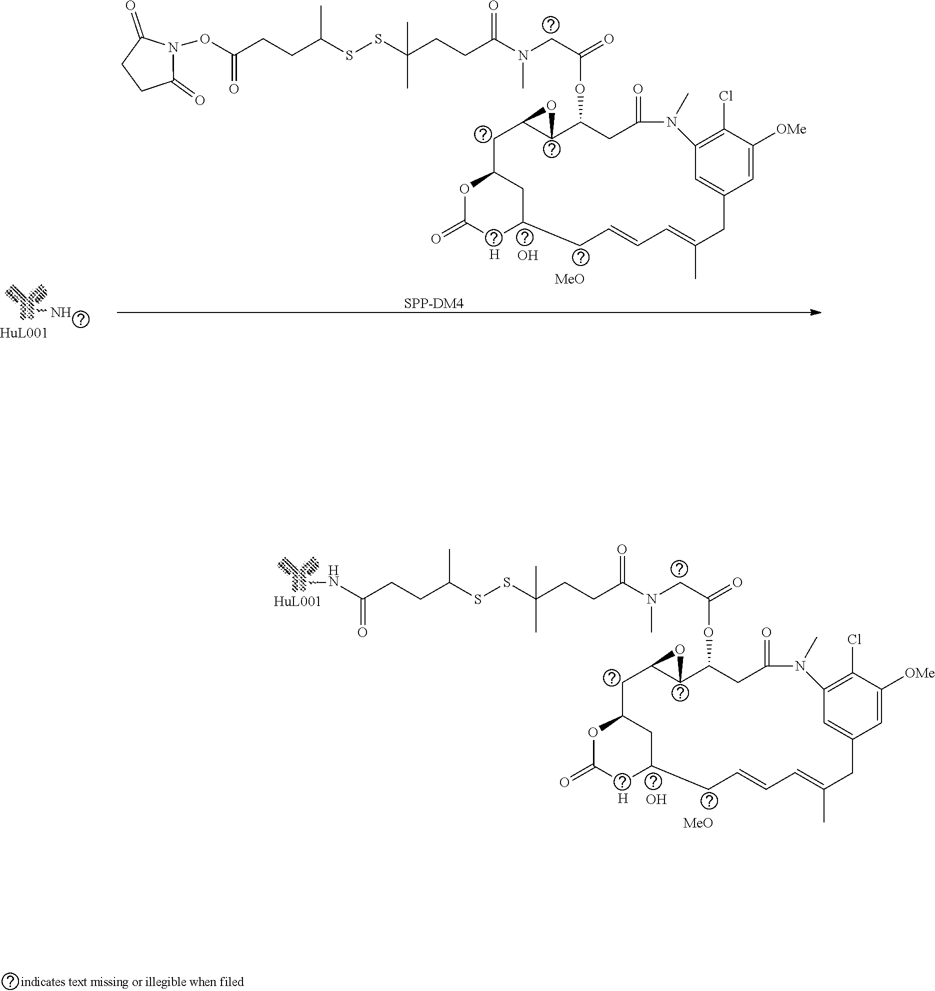

In this example, ADC contains DM4, which is another maytansine analog. DM4 is also a potent microtubule-targeted compounds that inhibit proliferation of cells at mitosis. Some embodiments of the invention may use DM4.

In this example, the buffer of HuL001 (70 mg) was exchanged to pH 6.5 Na-citrate buffer and adjust to 5 mg/mL. The solution of SPP-DM4 (10 mM in DMA, 15 eq) was slowly added to the HuL001 solution. The reaction mixture was incubated in shaking incubator (150 rpm) at 37° C. for 18 hours. The Amicon Ultra-15 centrifugal filter with 30 kDa NMWL and 25 mM Na-citrate pH 6.5 was used for the concentration of HuL001-SPP-DM4 and the removal of SPP-DM4. ADC concentration: 3.65 mg/mL, ADC yield: 34.3 mg (49%), and average DAR: 3.18.

### Example 4. Preparation of HuL001-mal-vc-MMAE Conjugates

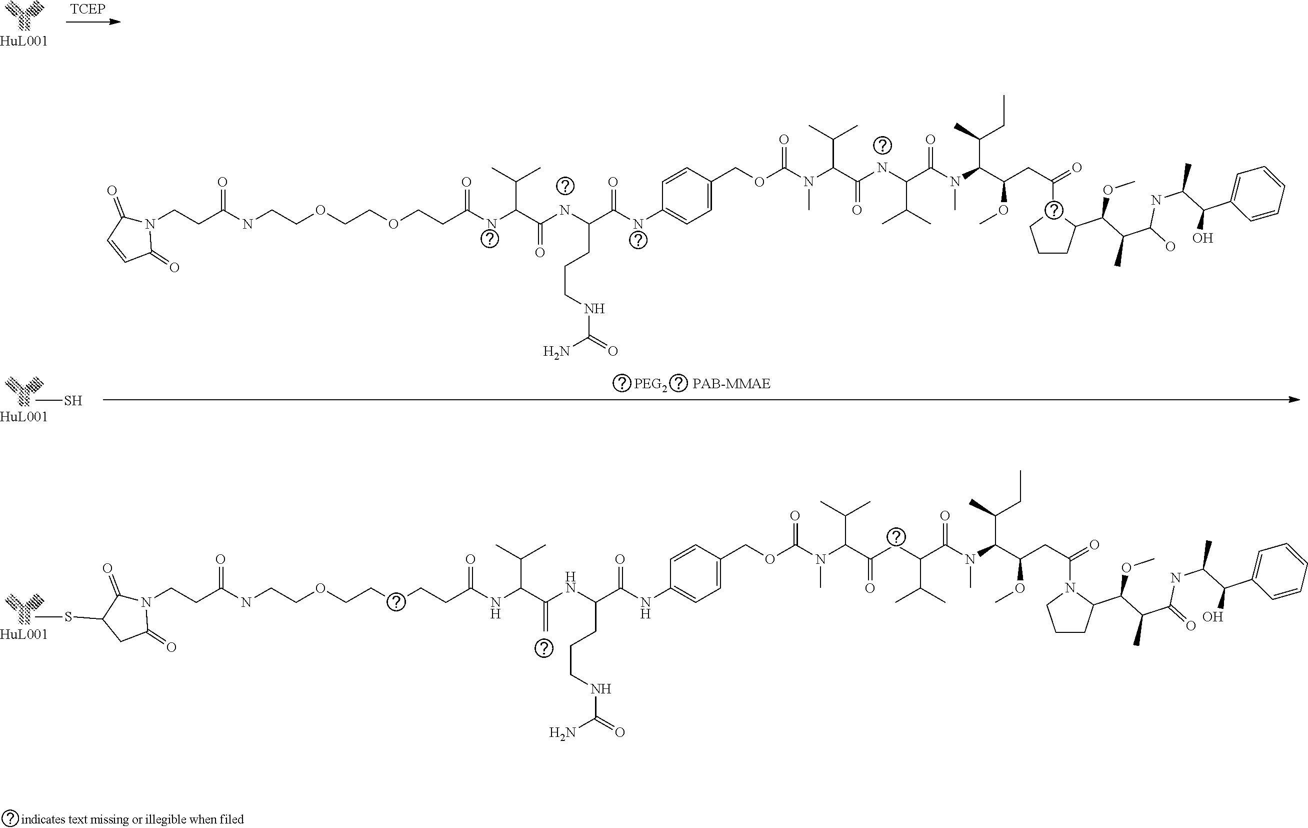

Monomethyl auristatin E (MMAE) is an antineoplastic agent, which inhibits cell division by blocking the polymerization of tubulins. It is derived from peptides occurring in marine shell-less mollusk (dolastatins). MMAE has been shown to be useful payloads for ADCs.

Linkers in ADCs may have significant impacts on the biological activities. For example, in vivo studies demonstrated that the peptide-linked conjugates induced regressions and cures of established tumor xenografts with therapeutic indices as high as 60-fold. These conjugates illustrate the importance of linker technology, drug potency and conjugation methodology in developing safe and efficacious mAb-drug conjugates for cancer therapy.

Some embodiments of the invention relate to MMAEs linked to antibodies via a lysosomally cleavable dipeptide, valine-citrulline (vc), which have been shown to improve ADC efficacies. In this example, the buffer of HuL001 (1 mg) was exchanged to pH 7.4 PBS/EDTA buffer and adjust to 5 mg/mL. The aqueous solution of TCEP (10 mM, 3 eq) was slowly added to the HuL001 solution. The disulfide bond in the antibody was reduced by incubating at 37° C. for 2 hours. Then the solution of mal-PEG2-vc-PAB-MMAE (10 mM in DMA, 10 eq) was slowly added to the protein solution. The reaction mixture was incubated in shaking incubator (150 rpm) at 25° C. for 2 hours. Then 100 mM NAC (20 eq) was used to quench the excess mal-vc-steroid. The Amicon Ultra-15 centrifugal filter with 10 kDa NMWL and buffer (pH 6.0 PBS and 137 mM NaCl) was used for the concentration of HuL001-mal-vc-MMAE and the removal of mal-PEG2-vc-PAB-MMAE. ADC concentration: 3.7 mg/mL, ADC yield: 0.481 mg (48.1%), and average DAR: 3.64.

### Example 5. Preparation of HuL001-Ph-MMAF Conjugates

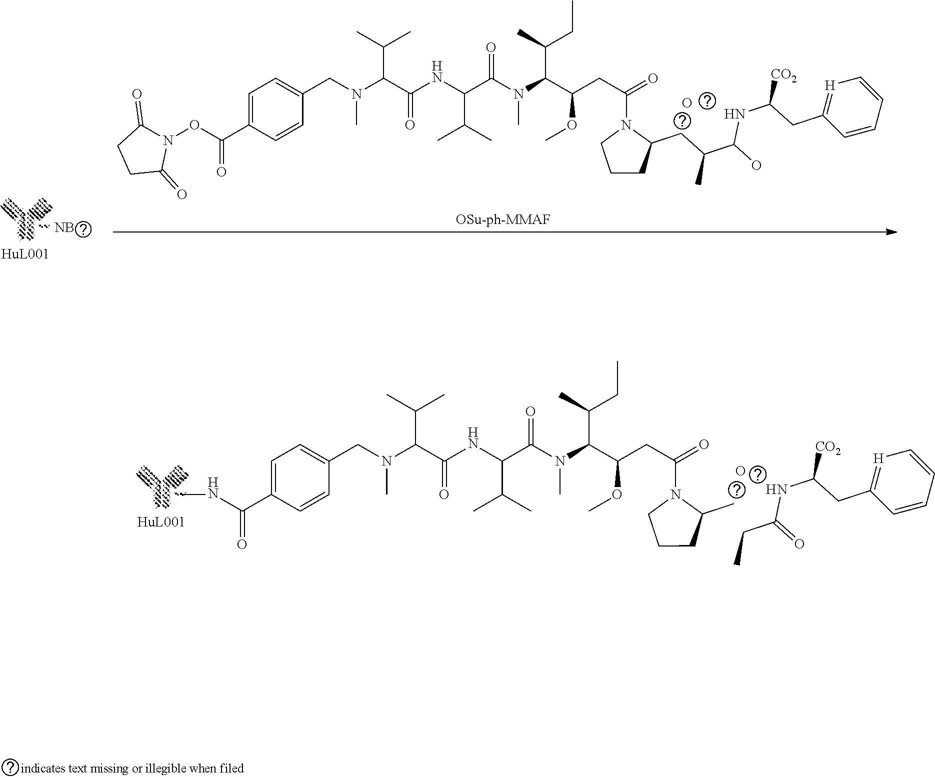

Some embodiments of the invention relate to ADCs containing Monomethyl auristatin F (MMAF), which is an analog of MMAE. The buffer of HuL001 (37 mg) was exchanged to pH 7.4 PBS buffer and adjust to 5 mg/mL. The solution of OSu-ph-MMAF (5 mM in DMA, 5 eq) was slowly added to the protein solution. The reaction mixture was incubated in shaking incubator (150 rpm) at 37° C. for 1 hours. The Amicon Ultra-15 centrifugal filter with 30 kDa NMWL and 25 mM Na-citrate pH 6.5 was used for the concentration of HuL001-ph-MMAF and the removal of OSu-ph-MMAF. ADC concentration: 7.8 mg/mL, ADC yield: 34.1 mg (92.1%), and average DAR: 3.09.

### Example 6. Preparation of HuL001-mal-vc-Steroid Conjugates

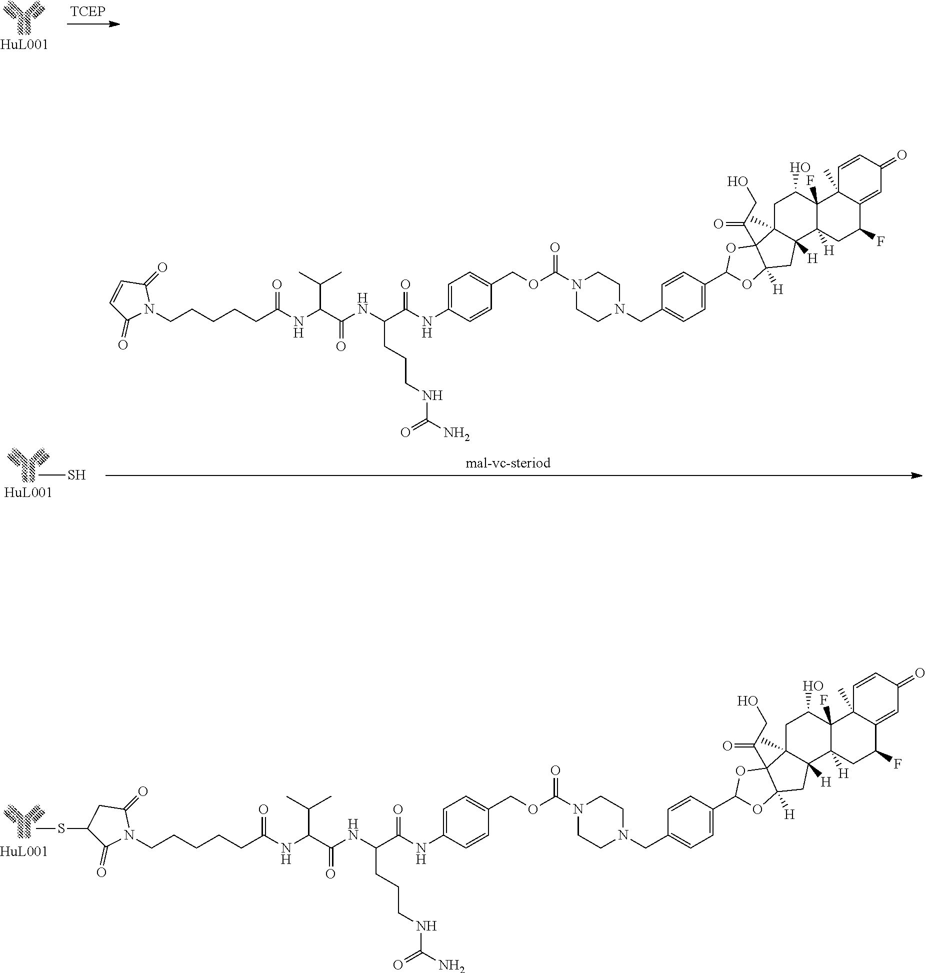

In this example, the buffer of HuL001 (1 mg) was exchanged to pH 7.4 PBS/EDTA buffer and adjust to 10 mg/mL. The aqueous solution of TCEP (10 mM, 4 eq) was slowly added to the protein solution. The disulfide bond in the antibody was reduced by incubating at 37° C. for 1.5 hours. Then the solution of mal-vc-steroid (10 mM in DMSO, 10 eq) was slowly added to the protein solution. The reaction mixture was incubated in shaking incubator (150 rpm) at 0° C. for 18 hours. Then 100 mM NAC (20 eq) was used to quench the excess mal-vc-steroid. The Amicon Ultra-15 centrifugal filter with 30 kDa NMWL and buffer (pH 6.0 PBS and 137 mM NaCl) was used for the concentration of HuL001-mal-vc-steroid and the removal of mal-vc-steroid. ADC concentration: 2.2 mg/mL, ADC yield: 0.33 mg (33%), and average DAR: 5.2.

### Example 7. PLRP-HPLC or HIC Analysis

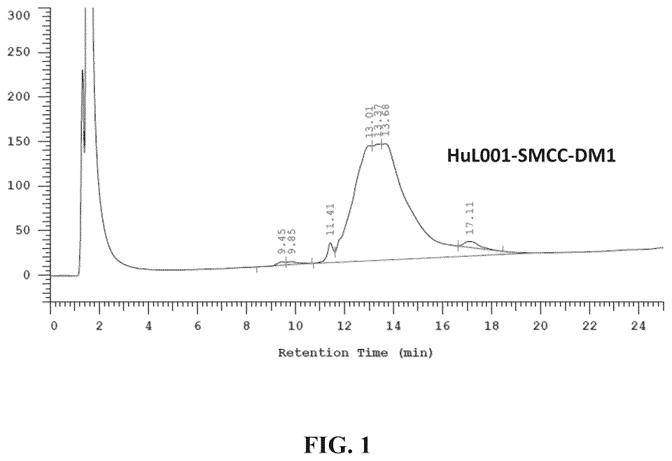

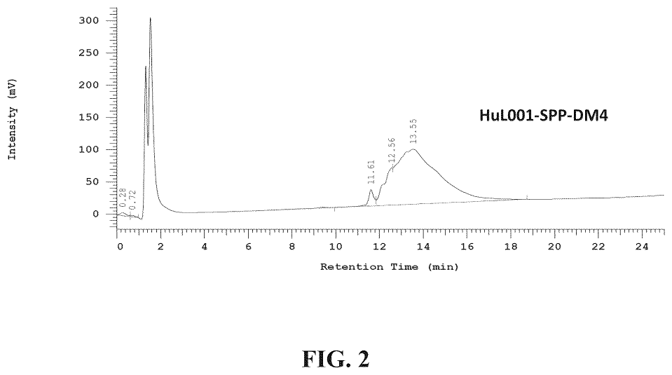

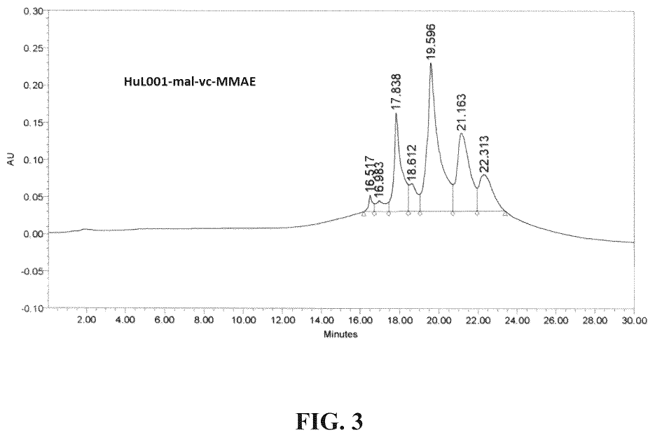

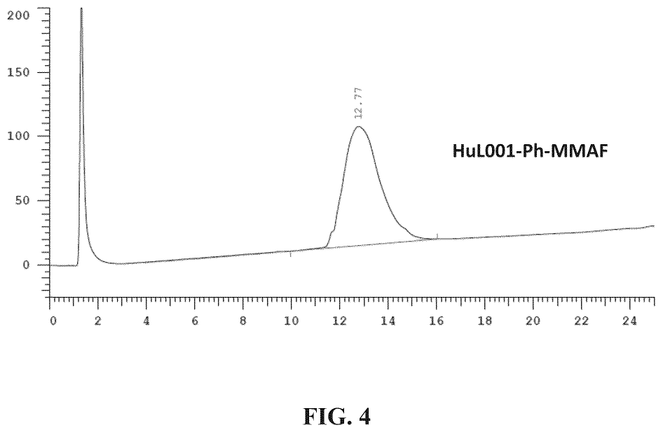

The various ADCs according to embodiments of the claimed invention was analyzed by PLRP-HPLC or HIC. FIGS. 1-5 show that the conjugation reactions were substantially completed and only residual amounts of anti-ENO-1 antibody and ADCs remained.

### Example 8. Intact or Reduced LC/MS Analysis

Evaluation the drug-to-antibody ratio (DAR) is important for monitoring of payload conjugation efficiency on target antibody. The drug-to-antibody ratio may affect the therapeutic efficacy of the anti-ENO-1 ADC products. Intact liquid chromatography-mass spectrometry (LC-MS) is the method of choice for determination of the drug-to-antibody ratio (DAR) and drug load distribution for lysine-linked antibody-drug conjugates (ADCs). Reduced LC-MS is the method of choice for determination of the DAR and drug load distribution for cysteine-linked ADCs. The area percentage of a peak represents the relative distribution of the particular drug-loaded ADC species. The weighted average DAR is then calculated using the percentage peak area information and the drug load numbers.

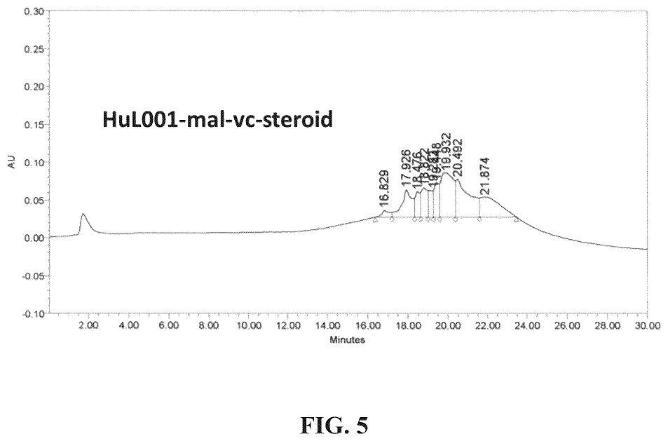

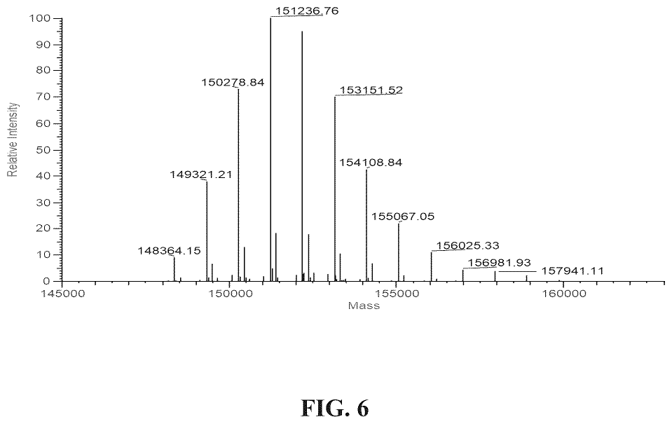

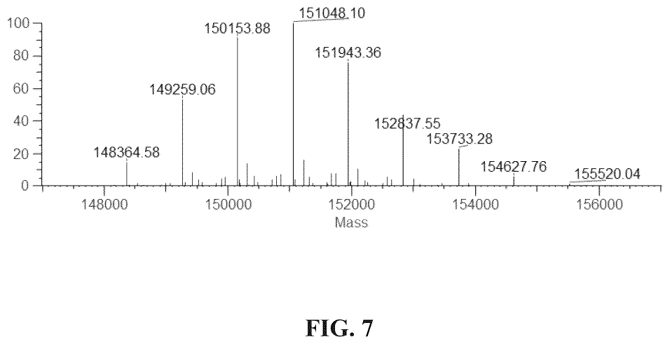

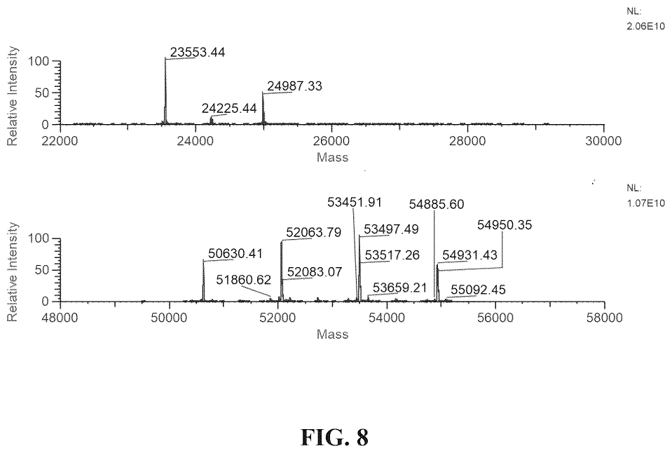

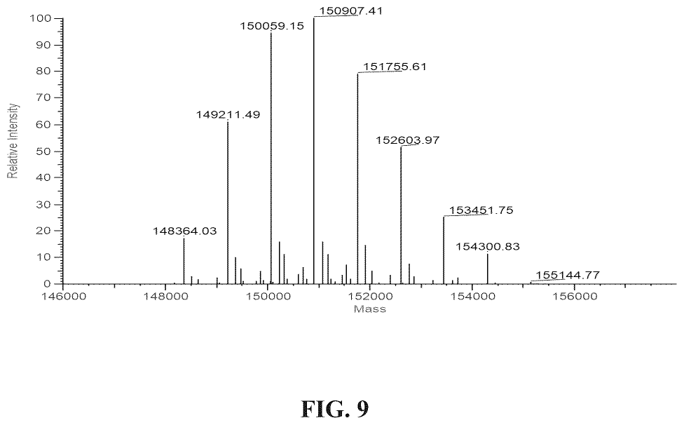

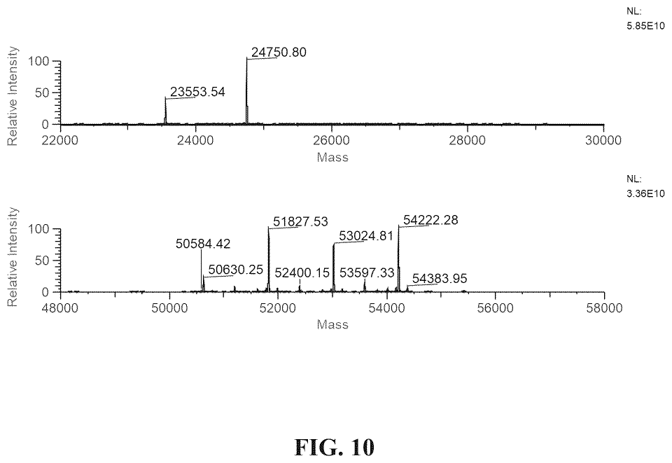

FIGS. 5-10 illustrate the examples of LC-MS analysis of an ADC according to embodiments of the claimed invention (anti-ENO-1 ADCs), which indicates a distribution of various numbers of drug attached to an antibody with the most abundant species having 1-12 drugs attached to an antibody. The range of the average drug-to-antibody ratio (DAR) in these examples are 3.18-5.2. Having multiple copies of the drug attached to one antibody would ensure more efficient delivery of the drug into cells.

### Example 9. Cytotoxicity Assay

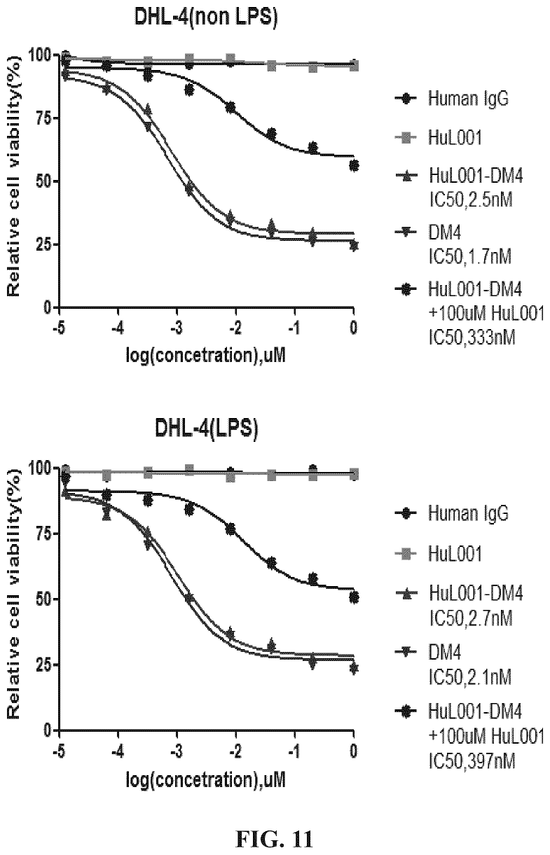

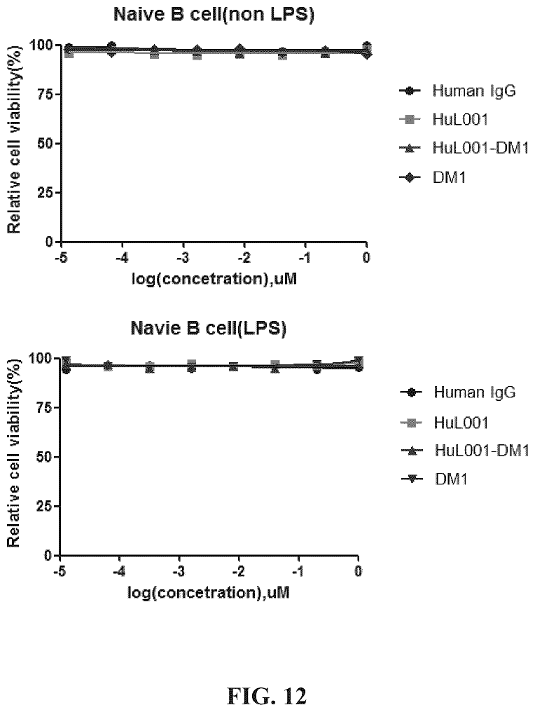

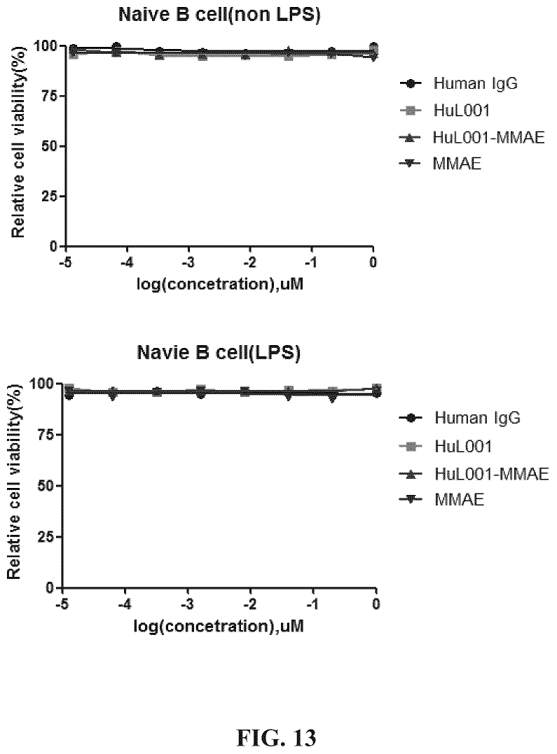

Cytokine-induced cytotoxicity effects of the anti-ENO-1 ADC products have been demonstrated in ENO-1-dependent cell lines that derived from different human cancer including lymphoma, lung cancer, breast cancer, pancreatic cancer and diffuse large B cell lymphoma (DLBCL). Various cell lines, for example, U937, A549, MDA-MB-231, MCF-7, MBA-MD-453, MBA-MD-175, PANC-1, Raji, SU-DHL-4, Toledo, GA-10, or HT were activated by cytokines TGF-β, MCP-1, or IL-6, respectively, for 4 hours to mimic inflammatory tumor microenvironment, and incubated with serial diluted antibody solution for another 72 hours. The cell viability was measured by using cell counting (CCK-8) kit and the values of IC₅₀ were calculated. The IC₅₀ of HuL001 for the cells tested is above the highest concentration (1000 nM) tested. The results indicated that the anti-ENO-1 ADC products specifically suppress cytokines-induced (20 ng/mL TGF-β, 100 ng/ml MCP-1, 50 ng/ml IL-6) pro-inflammatory surface ENO-1 from various cell lines. The Table 1 and Table 2 indicate that in most cell lines, the IC₅₀ of HuL001-SMCC-DM1 and HuL001-mal-vc-MMAE decrease dramatically to single digital level after activation to reflect the cytotoxicity effect. The FIG. 11 indicates the single digital level of IC₅₀ of HuL001-SPP-DM4 in DLBCL cell line DHL-4. The FIG. 12 and FIG. 13 indicate no detectable in vitro cytotoxicity of either HuL001 or HuL001-SMCC-DM1 and HuL001-mal-vc-MMAE in isolated normal human B cells in the presence or absence of LPS stimulation. In summary, the ADC according to the present disclosure effectively kills the cancer cells, especially upon cytokine simulation, without affecting the viability of the normal cells. It is considered a potential candidate for the targeted cancer therapy.

TABLE 1
in vitro cytotoxicity results of HuL001-SMCC-
DM1 in cytokine-stimulated cancer cell lines

| IC₅₀ (nM) of HuL001-SMCC- | | | | | |
|---|---|---|---|---|---|
| DM1-induced cytotoxicity | | | | IC₅₀ of HuL001 is >1000 nM | |
| | | Untreated | TGF-β | MCP-1 | IL-6 |
| Monocyte | U937 | 75 ± 5.65 | 7.5 | 9.7 | 7.1 |
| Lung cancer | A549 | 56 ± 4.38 | 8.2 | 12.1 | 11.2 |
| Breast cancer | MDA-MB-231 | 68.95 ± 7.71 | 24.2 | 22.4 | 34.6 |
| | MCF-7 | 74.9 | 53.5 | 36.2 | 38.6 |
| | MDA-MB-453 | 39.8 | 16.6 | 24.7 | 22.8 |
| | MDA-MB-175 | 76.4 | 38.3 | 40.5 | 45.1 |
| Pancreatic cancer | PANC-1 | 174 | 116 | 109.6 | 134.3 |
| Diffuse Large B- | Raji | 32.2 ± 3.39 | 18.6 | 21.7 | 17.1 |
| Cell Lymphoma | SU-DHL-4 | 7.65 ± 0.91 | 7.4 | 7.6 | 8.3 |
| | Toledo | 8 | 9.8 | 8.1 | 11.4 |
| | DB | 5.5 | 5.1 | 4.1 | 5.8 |
| | GA-10 | 37.8 | 22.8 | 25.6 | 26.6 |
| | HT | 7.3 | 6.1 | 7.6 | 6.6 |

TABLE 2
in vitro cytotoxicity results of HuL001-mal-vc-
MMAE in cytokine-stimulated cancer cell lines

| IC₅₀ (nM) of HuL001-mal-vc- | | | | | |
|---|---|---|---|---|---|
| MMAE-induced cytotoxicity | | | | IC₅₀ of HuL001 is >1000 nM | |
| | | Untreated | TGF-β | MCP-1 | IL-6 |
| Monocyte | U937 | 29.2 | 5.3 | 6.7 | 3.7 |
| Lung cancer | A549 | 32.9 | 7.3 | 8.3 | 7.8 |
| Breast cancer | MDA-MB-231 | 75.3 | 32.4 | 45.1 | 35.8 |
| | MDA-MB-453 | 95.2 | 40.5 | 38.3 | 31.8 |
| Diffuse Large B- | Raji | 32.4 | — | — | — |
| Cell Lymphoma | SU-DHL-4 | 4.5 | — | — | — |
| | Toledo | 9.7 | — | — | — |
| | DB | 5.3 | — | — | — |
| | GA-10 | 37.1 | — | — | — |
| | HT | 6.9 | 7.5 | 7.5 | 7.7 |

### Example 10. Cytokine Assay

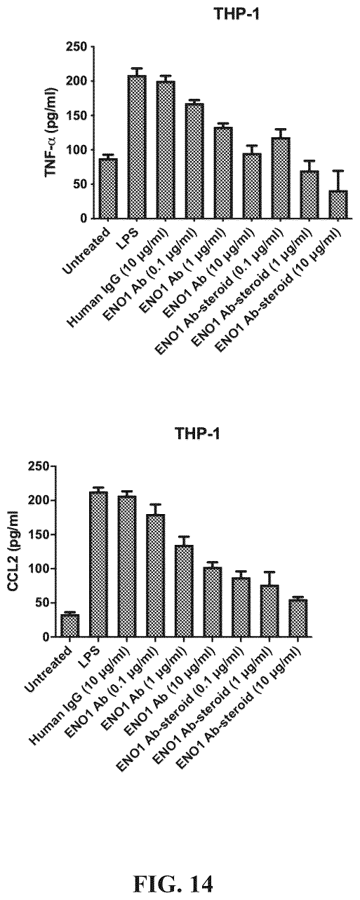

Anti-inflammatory effects of the anti-ENO-1-steroid ADC was demonstrated in human monocyte cell line THP-1. THP-1 cells were stimulated with LPS to induce ENO-1 surface expression and secretion of pro-inflammatory cytokines TNF-α and CCL2. FIG. 14 indicates superior anti-inflammatory effect of anti-ENO-1-steroid ADC compared to anti-ENO-1 antibody.

### Example 11. Prostate Cancer Model of Anti-ENO-1 ADC

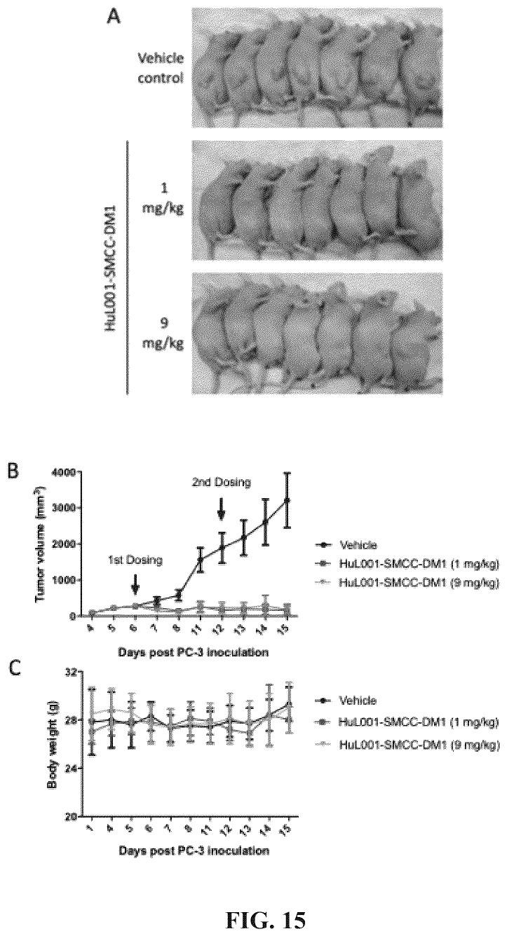

HuL001-SMCC-DM1 was evaluated in experimental castration-resistant prostate cancer model. Male 4˜6-week-old nude (nu/nu) mice were used (Lasco Co., Ltd., Taiwan). Before inoculation, castration-resistant human prostate cancer cell line PC-3 cells were washed with PBS and resuspended with PBS and Matrigel at 1:1 for final concentration of 10⁷ cells/ml. Cells (10⁶/100 μl) were implanted subcutaneously into the right flank of mice. After average tumor volume reached 100 mm³ (6 days post implantation), the mice were randomized to control and treatment groups, which were administrated with PBS (5 ml/kg) as vehicle control or HuL001-SMCC-DM1 (1 or 9 mg/kg), respectively. HuL001-SMCC-DM1 was administrated by intraperitoneal injection once by every 6 days for total of 2 dosing until the end of study. Body weight and tumor volume measurement were performed daily. The volume of subcutaneous tumor was determined according to the formula: tumor volume=shorter diameter²×longer diameter/2. was dosed by intra-peritoneal injection. FIG. 15 indicates that HuL001-SMCC-DM1 inhibits tumor growth without inducing body weight loss.

### Example 12. EAE Disease Model of Anti-ENO-1 ADC

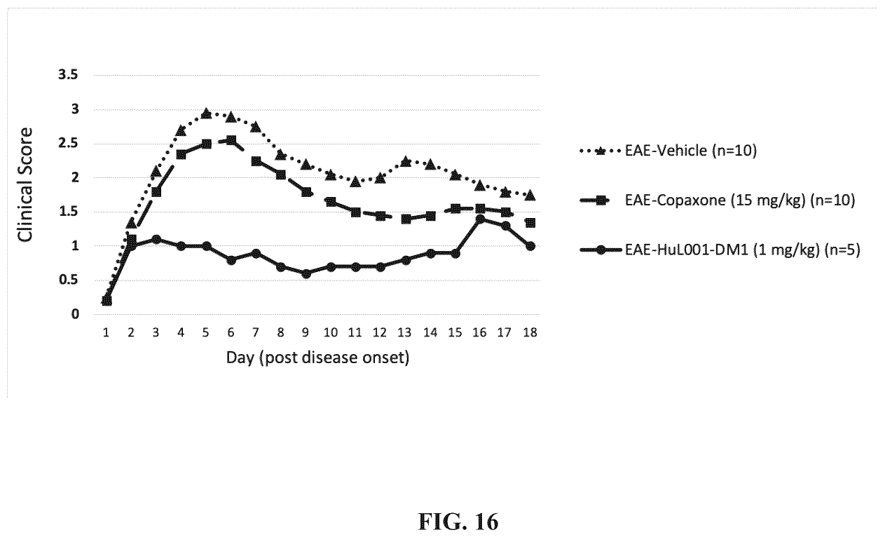

HuL001-SMCC-DM1 was evaluated in experimental autoimmune encephalomyelitis (EAE) in C57BL/6 mice which are the most commonly used experimental models for the human inflammatory demyelinating disease, multiple sclerosis. 10 to 12-week-old female C57BL/6 mice were provided subcutaneously with 100 micrograms of MOG p 35-55 in Freud's complete adjuvant, and then 100 ng of pertussis toxin was injected intraperitoneally. On the second day, another dose of 100 ng of pertussis toxin was administered. Animal were observed daily and the clinical symptoms were assessed as follows: 0, no sign; decreased tail tone; 2, mild monoparesis or paresis; 3, severe paraparesis; 4, paraplegia and or quad-paraparesis; 5 moribund or death. Mice were randomly divided into 3 groups with 10 mice in control and COPAXONE® (Teva pharmaceuticals), and 5 mice in HuL001-SMCC-DM1 group. The day of disease onset (disease score >1) was set as day 1. EAE mice of HuL001-SMCC-DM1 group were injected subcutaneously on day 1, 8, and 15. EAE mice of COPAXONE group were injected subcutaneously every day from day 1 to day 18. FIG. 16 illustrates treating with HuL001-SMCC-DM1 was able to slow the progression of disease symptoms of EAE mice compared with the PBS control group even COPAXONE® group.

### Example 13. IPF Disease Model of Anti-ENO-1 ADC

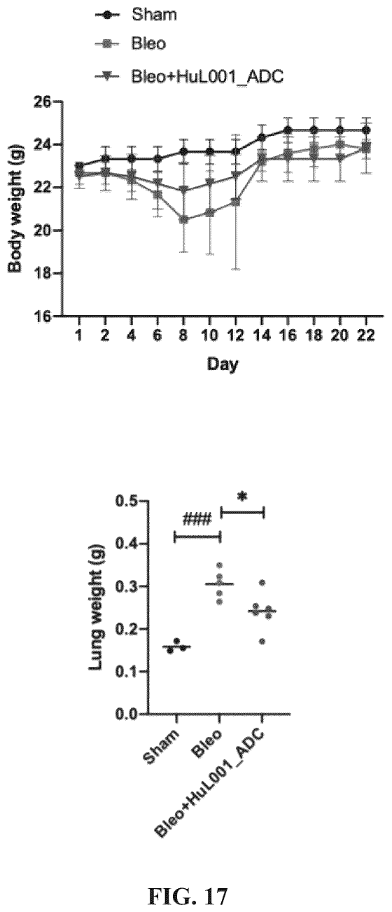

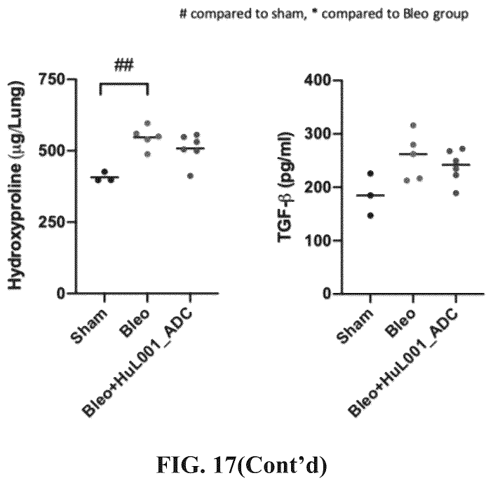

HuL001-SMCC-DM1 was evaluated in bleomycin-induced pulmonary fibrosis in C57BL/6 mice which are the most commonly used experimental models for the human fibrotic disease, idiopathic pulmonary fibrosis. 7 to 9-week-old male C57BL/6 mice were intra-tracheally given with single dosing of bleomycin (3 mg/kg). Mice were randomly divided into 3 groups with 3 mice in sham group, and 6 mice in Bleomycin and HuL001-SMCC-DM1 groups, respectively. The day of bleomycin challenge was set as day 0. Mice of HuL001-SMCC-DM1 group were injected subcutaneously on day 1, 8, and 15. FIG. 17 illustrates treating with HuL001-SMCC-DM1 was able to attenuate body weight loss and lung weight gain compared with the bleomycin group. Lung hydroxyproline content and TGF-β levels in bronchial alveolar lavage fluid (BALF) were reduced by HuL001-SMCC-DM1 treatment.

Unless defined otherwise, all technical and scientific terms and any acronyms used herein have the same meanings as commonly understood by one of ordinary skill in the art in the field of this invention. Although any compositions, methods, kits, and means for communicating information similar or equivalent to those described herein can be used to practice this invention, the preferred compositions, methods, kits, and means for communicating information are described herein.

All references cited herein are incorporated herein by reference to the full extent allowed by law. The discussion of those references is intended merely to summarize the assertions made by their authors. No admission is made that any reference (or a portion of any reference) is relevant prior art. Applicants reserve the right to challenge the accuracy and pertinence of any cited reference.

1. An immunoconjugate that binds specifically to ENO-1, comprising:
the general formula of Ab-(L-D)ₘ (I),
wherein Ab is the anti-ENO-1 antibody or the binding fragment thereof, L is a linker or a direct bond, D is a therapeutic agent or a label, and m is an integer from 1 to 12.

2. The immunoconjugate according to claim 1, wherein the antibody is a monoclonal antibody.

3. The immunoconjugate according to claim 1, wherein the antibody is a mouse antibody, a human antibody, a chimeric antibody, a humanized antibody, or an antibody fragment thereof.

4. The immunoconjugate according to claim 1, wherein the therapeutic agent comprises a cytotoxic agent, immunomodulator, radioactive isotopes, and toxins.

5. The immunoconjugate according to claim 1, wherein the cytotoxic agent comprises maytansinoid 1 (DM1), maytansinoid 4 (DM4), Monomethyl auristatin E (MMAE), Monomethyl auristatin F (MMAF), anthracycline, pyrrolobenzodiazepine, α-amanitin, tubulysin, benzodiazepine, erlotinib, bortezomib, fulvestrant, sunitinib, letrozole, imatinib mesylate, PTK787/ZK 222584, oxaliplatin, leucovorin, rapamycin, lapatinib, lonafarnib, sorafenib, gefitinib, AG1478, AG1571, alkylating agents including thiotepa or cyclosphosphamide; alkyl sulfonates including busulfan, improsulfan or piposulfan; aziridines including benzodopa, carboquone, meturedopa, or uredopa; ethylenimines and methylamelamines including altretamine, triethylenemelamine, triethylenephosphoramide, triethylenethiophosphoramide and trimethylomelamine; acetogenins; a camptothecin; bryostatin; callystatin; CC-1065; cryptophycins; dolastatin; duocarmycin; eleutherobin; pancratistatin; a sarcodictyin; spongistatin; nitrogen mustards including chlorambucil, chlornaphazine, cholophosphamide, estramustine, ifosfamide, mechlorethamine, mechlorethamine oxide hydrochloride, melphalan, novembichin, phenesterine, prednimustine, trofosfamide, or uracil mustard; nitrosureas including carmustine, chlorozotocin, fotemustine, lomustine, nimustine, or ranimustine; antibiotics including the enediyne antibiotics including calicheamicin, especially calicheamicin gamma1I and calicheamicin omegaI1; dynemicin, including dynemicin A; bisphosphonates including clodronate; an esperamicin; as well as neocarzinostatin chromophore and related chromoprotein enediyne antibiotic chromophores, aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin, epirubicin, esorubicin, idarubicin, marcellomycin, mitomycins including mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites including methotrexate and 5-fluorouracil (5-FU); folic acid analogues including denopterin, pteropterin, trimetrexate; purine analogs including fludarabine, 6-mercaptopurine, thiamiprine, thioguanine; pyrimidine analogs including ancitabine, azacitidine, 6-azauridine, carmofur, cytarabine, dideoxyuridine, doxifluridine, enocitabine, floxuridine; androgens including calusterone, dromostanolone propionate, epitiostanol, mepitiostane, testolactone; anti-adrenals including aminoglutethimide, mitotane, trilostane; folic acid replenisher including frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids including maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; polysaccharide complex; razoxane; rhizoxin; sizofiran; spirogermanium; tenuazonic acid; triaziquone; 2,2′,2″-trichlorotriethylamine; trichothecenes; urethan; vindesine; dacarbazine; mannomustine; mitobronitol; mitolactol; pipobroman; gacytosine; arabinoside; cyclophosphamide; thiotepa; taxoids, paclitaxel, albumin-engineered nanoparticle formulation of paclitaxel, and doxetaxel; chloranbucil; gemcitabine; 6-thioguanine; mercaptopurine; methotrexate; platinum analogs including cisplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids including retinoic acid; capecitabine; and pharmaceutically acceptable salts, acids or derivatives of any of the above.

6. The immunoconjugate according to claim 1, wherein the label comprises a diagnostic or imaging reagent.

7. The immunoconjugate according to claim 1, wherein the antibody comprises:
a heavy-chain variable domain having three complementary regions including
HCDR1
(GYTFTSCVMN; SEQ ID NO: 1),
HCDR2
(YINPYNDGTKYNEKFKG; SEQ ID NO: 2),
and
HCDR3
(EGFYYGNFDN; SEQ ID NO: 3);
and
a light-chain variable domain having three complementary regions including
LCDR1
(RASENIYSYLT; SEQ ID NO: 4),
LCDR2
(NAKTLPE; SEQ ID NO: 5),
and
LCDR3
(QHHYGTPYT; SEQ ID NO: 6).

8. The immunoconjugate according to claim 1, wherein the antibody comprises:
a heavy-chain variable domain having three complementary regions including
HCDR1
(GYTFTSXVMN,
wherein X is any amino acid but
cysteine; SEQ ID NO: 7),
HCDR2
(YINPYNDGTKYNEKFKG; SEQ ID NO: 2),
and
HCDR3
(EGFYYGNFDN; SEQ ID NO: 3);
and
a light-chain variable domain having three complementary regions including
LCDR1
(RASENIYSYLT; SEQ ID NO: 4),
LCDR2
(NAKTLPE; SEQ ID NO: 5), and
LCDR3
(QHHYGTPYT; SEQ ID NO: 6).

9. A composition for diagnosing or imaging cells or a tissue expressing ENO-1, comprising the immunoconjugate according to claim 6.

10. A pharmaceutical composition for use in treating an inflammatory disease, an immune disorder, or an ENO-1-expressing cancer, comprising the immunoconjugate according to claim 1.

11. The pharmaceutical composition according to claim 10, wherein the antibody comprises:
a heavy-chain variable domain having three complementary regions including
HCDR1
(GYTFTSCVMN; SEQ ID NO: 1),
HCDR2
(YINPYNDGTKYNEKFKG; SEQ ID NO: 2)
and
HCDR3
(EGFYYGNFDN; SEQ ID NO: 3),
and
a light-chain variable domain having three complementary regions including
LCDR1
(RASENIYSYLT; SEQ ID NO: 4),
LCDR2
(NAKTLPE; SEQ ID NO: 5)
and
LCDR3
(QHHYGTPYT; SEQ ID NO: 6).

12. The pharmaceutical composition according to claim 10, wherein the antibody comprises:
a heavy-chain variable domain having three complementary regions including
HCDR1
(GYTFTSXVMN,
wherein X is any amino acid
but cysteine; SEQ ID NO: 7),
HCDR2
(YINPYNDGTKYNEKFKG; SEQ ID NO: 2)
and
HCDR3
(EGFYYGNFDN; SEQ ID NO: 3),
and
a light-chain variable domain having three complementary regions including
LCDR1
(RASENIYSYLT; SEQ ID NO: 4),
LCDR2
(NAKTLPE; SEQ ID NO: 5)
and
LCDR3
(QHHYGTPYT; SEQ ID NO: 6).

13. The pharmaceutical composition of claim 10, wherein the inflammatory disease or the immune disorder comprises multiple sclerosis, rheumatoid arthritis, Crohn's disease, ulcerative colitis, systemic lupus erythematosus, chronic obstructive pulmonary disease (COPD), atopic dermatitis, idiopathic pulmonary fibrosis, nonalcoholic steatohepatitis, asthma, allergy, psoriasis, psoriatic arthritis, type 1 diabetes mellitus, atherosclerosis, osteoporosis, systemic sclerosis, virus induced pneumonia, or macrophage activation syndrome.

14. The pharmaceutical composition of claim 10, wherein the cancer comprises leukemia, multiple myeloma, gastric carcinoma, skin cancer, lung cancer, melanoma, renal cancer, liver cancer, myeloma, prostate cancer, breast cancer, colorectal cancer, gastric cancer, pancreatic cancer, thyroid cancer, hematological cancer, lymphoma, leukemia, skin cancer, ovarian cancer, bladder cancer, urothelial carcinoma, head and neck cancer, or metastatic lesion(s) of the cancer.

15. A method for treating an inflammatory disease, an immune disorder or a cancer, comprising administering to a subject in need thereof an effective amount of the immunoconjugate of claim 1.

16. (canceled)

17. A method for treating an inflammatory disease, an immune disorder or a cancer, comprising administering to a subject in need thereof an effective amount of the pharmaceutical composition of claim 10.


SEQUENCE LISTING (7 sequences)

SEQ ID NO: 1 - protein, 10 aa, Artificial Sequence
  HCDR1
GYTFTSCVMN

SEQ ID NO: 2 - protein, 17 aa, Artificial Sequence
  HCDR2
YINPYNDGTKYNEKFKG

SEQ ID NO: 3 - protein, 10 aa, Artificial Sequence
  HCDR3
EGFYYGNFDN

SEQ ID NO: 4 - protein, 11 aa, Artificial Sequence
  LCDR1
RASENIYSYLT

SEQ ID NO: 5 - protein, 7 aa, Artificial Sequence
  LCDR2
NAKTLPE

SEQ ID NO: 6 - protein, 9 aa, Artificial Sequence
  LCDR3
QHHYGTPYT

SEQ ID NO: 7 - protein, 10 aa, Artificial Sequence
  HCDR1
  MUTAGEN (7)..(7): Xaa is any amino acid but cysteine
GYTFTSXVMN